(19) United States
(12) Patent Application Publication
(10) Pub. No.: US 2009/0018596 A1
(43) Pub. Date: Jan. 15, 2009

(54) BAROREFLEX ACTIVATION THERAPY DEVICE WITH PACING CARDIAC ELECTRICAL SIGNAL DETECTION CAPABILITY

(75) Inventor: Robert S. Kieval, Medina, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Minneapolis, MN (US)

(21) Appl. No.: 12/120,356

(22) Filed: May 14, 2008

Related U.S. Application Data
(60) Provisional application No. 60/938,082, filed on May 15, 2007.

(51) Int. Cl. A61N 1/365

(52) U.S. Cl. 607/17

(57) ABSTRACT

An exemplary embodiment of the present invention provides systems, devices, and methods for using the same for activating (stimulating) the baroreflex system of a patient using a baroreflex activation system with pacing cardiac electrical signal detection capability.

### CROSS-REFERENCES TO RELATED APPLICATIONS

The present application claims the benefit of provisional U.S. Application No. 60/938,082 (Attorney Docket No. 021433-002900US), filed May 15, 2007, the full disclosure of which is incorporated herein by reference.

This application is related to but does not claim the benefit of U.S. Pat. Nos. 6,522,926 filed on Sep. 27, 2000 and 6,616,624 filed on Oct. 30, 2000. This application is also related to PCT Patent Application No. PCT/US01/30249 filed Sep. 27, 2001 (Attorney Docket No. 021433-000140PC) and the following U.S. patent application Ser. Nos. 09/964,079 (Attorney Docket No. 021433-000110US) filed on Sep. 26, 2001; 09/963,777 (Attorney Docket No. 021433-000120US) filed Sep. 26, 2001; 09/963,991 (Attorney Docket No. 021433-000130US) filed Sep. 26, 2001; 10/284,063 (Attorney Docket No. 021433-000150US) filed Oct. 29, 2002; 10/453,678 (Attorney Docket No. 021433-000210US) filed Jun. 2, 2003; 10/402,911 (Attorney Docket No. 021433-000410US) filed Mar. 27, 2003; 10/402,393 (Attorney Docket No. 021433-000420US) filed Mar. 27, 2003; 10/818,738 (Attorney Docket No. 021433-000160US) filed Apr. 5, 2004; 60/584,730 (Attorney Docket No. 021433-001200US) filed Jun. 30, 2004; 10/958,694 (Attorney Docket No. 021433-001600US) filed Oct. 4, 2004; 60/882,478 (Attorney Docket No. 021433-002400US) filed Dec. 28, 2006; 60/883,721 (Attorney Docket No. 021433-002500US) filed Jan. 5, 2007; and 60/894,957 (Attorney Docket No. 021433-002600US) filed Mar. 15, 2007. All of the above patents and applications are hereby incorporated fully by reference in their entirety.

### BACKGROUND OF THE INVENTION

The present invention relates generally to medical devices and methods of use for the treatment and/or management of cardiovascular, neurological, and renal disorders, and more specifically to devices and methods for controlling the baroreflex system for the treatment and/or management of cardiovascular, neurological, and renal disorders and their underlying causes and conditions in combination with cardiac rhythm management devices (“CRMD”), such as those used for providing cardiac resynchronization (CRT) or treatment of arrhythmia to treat heart failure.

Hypertension, or high blood pressure, is a major cardiovascular disorder that is estimated to affect 65 million people in the United States alone, and is a leading cause of heart failure and stroke. It is listed as a primary or contributing cause of death in over 200,000 patients per year in the United States alone. Hypertension occurs in part when the body's smaller blood vessels (arterioles) constrict, causing an increase in blood pressure. Because the blood vessels constrict, the heart must work harder to maintain blood flow at the higher pressures. Sustained hypertension may eventually result in damage to multiple body organs, including the kidneys, brain, eyes and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke.

Congestive heart failure (CHF) is an imbalance in pump function in which the heart fails to maintain the circulation of blood adequately. The most severe manifestation of CHF, pulmonary edema, develops when this imbalance causes an increase in lung fluid due to leakage from pulmonary capillaries into the lung. More than 3 million people have CHF, and more than 400,000 new cases present yearly. Prevalence of CHF is 1-2% of the general population. Approximately 30-40% of patients with CHF are hospitalized every year. CHF is the leading diagnosis-related group (DRG) among hospitalized patients older than 65 years. The 5-year mortality rate after diagnosis of CHF is around 60% in men and 45% in women.

A number of different treatment modalities may be attempted for treating heart failure, such as medications, mechanical restriction of the heart, surgical procedures to reduce the size of an expanded heart and the like.

One CHF treatment method that has been proposed is to affect the baroreflex system to help the heart perform more efficiently by way of controlling the patient's blood pressure. Baroreflex activation may generally decrease neurohormonal activation, thus decreasing cardiac afterload, heart rate, sympathetic drive to the heart, and the like. By decreasing the demands placed on the heart, baroreflex activation may help prevent or treat CHF.

It would be desirable to provide improved methods and devices for treating heart failure. Ideally, such methods and apparatus would be minimally invasive, with few if any significant side effects. Ideally, one or more underlying mechanisms causing heart failure could be treated in some cases. Additionally, it would be desirable to have methods and devices for activation of the baroreflex system of the patient at a time when the body expects such activation to enhance the efficacy of the baroreflex activation therapy. Furthermore, it would be desirable to provide improved methods and systems to reduce the likelihood of ventricular fibrillation. At least some of these objectives will be met by the present invention.

### BRIEF SUMMARY OF THE INVENTION

To address the problems of hypertension, heart failure, other cardiovascular disorders, nervous system and renal disorders, the present invention provides methods, devices, and systems for stimulating the baroreflex system of a patient's body. In some embodiments, the baroreflex activation devices and methods are used for treating patients who are furthermore under treatment with a cardiac rhythm management (CRM) device in conjunction with a baroreflex activation device for activating at least one baroreflex system within a patient's body. These effects may include reducing excessive blood pressure. In some exemplary embodiments, baroreflex activation therapy (BAT) suggests to the brain that the body is experiencing an increase in blood pressure. This suggestion may cause the brain to regulate (e.g., decrease) the level of sympathetic nervous system and neurohormonal activation. In some cases, the brain may also increase the level of sympathetic nervous system activity. These reactions may reduce blood pressure and have additional beneficial effects on the cardiovascular system and other body systems.

As used herein, for convenience, the term “baroreceptor” will refer to collectively, receptors, including baroreceptors, mechanoreceptors, pressoreceptors, or any other venous heart, or cardiopulmonary receptors which affect the blood pressure, nervous system activity, and neurohormonal activity in a manner analogous to baroreceptors in the arterial vasculation. The activation of the baroreflex system may also be affected by stimulating nerves which carry signals from such baroreceptors. As used herein, the term “baroreflex activation device” means a device that is located at or near a baroreceptor, so as to activate the baroreflex system within the patient's body. For the purposes of discussions, the present invention will be further explained referring to baroreceptors, but that is not intended to limit the scope of the present invention and applies to nerves (e.g., as referenced above). Furthermore, the present invention, although will be mainly discussed in reference to baroreflex activation systems, baroreflex devices, and implantable pulse generators in the context of such systems and devices, it is not meant to be limiting. In some embodiments, the activation of the nerve or receptor causes a baroreflex response in a patient. The activation of the baroreflex response may be by way of stimulating a baroreceptor or a nerve leading from a baroreceptor.

In some exemplary embodiments, both the baroreflex activation device and the cardiac rhythm management device are combined as a single implantable device, while in others, they are housed in different housings and separate devices which are configured for communication with one another. Such communication may be by way of hard wiring or by way of telemetry as is known in the art. Additionally, the baroreflex activation device may be a closed loop device where it controls a baroreflex therapy as previously pre-programmed, open loop (e.g., controllable by the user such as the patient or the health-care provider), or both (e.g., closed loop with possibility of intervention by the user).

In some exemplary embodiments, the baroreflex activation system is configured to adjust its baroreflex therapy based on actively detecting (i.e., pulling of information) from a cardiac rhythm management device and thereafter providing or adjusting the stimulation of the baroreflex system of the patient.

In an exemplary embodiment, a method for treating a patient includes stimulating a baroreflex system of the patient with a baroreflex activation device according to a baroreflex activation therapy (“BAT”), detecting a pacing pulse generated from a cardiac management device (“CRM”) device by the baroreflex activation device; and adjusting the baroreflex activation therapy (“BAT”) in response to the detected pacing pulse as necessary. The stimulation, may generally, be achieved by stimulating a nerve or receptor within the human body. The nerve or receptor may be a baroreceptor or a nerve leading from a baroreceptor.

The intensity of the BAT may be adjusted in response to the frequency of the detected pacing pulse. The BAT may be performed in one or multiple steps. A pre-determined time delay may be selected by the patient/healthcare provider or the device itself (e.g., by way of its algorithms) between applying the BAT. The baroreflex activation therapy may be delivered at a pre-selected time after detecting pacing pulses generated by the CRM device. Alternatively, the baroreflex therapy may be delivered during either of systole or diastole cardiac phases, more likely during which the patient's response to such therapy is more effective. The amount of time delay may change, as by way of example, it may decrease as the frequency of detected pacing pulses increases.

Generally, the baroreflex activation device is connectable to an output terminal of the pacing pulse generator CRM device. The baroreflex activation device may calculate the level of adjustment of the baroreflex activation therapy based on the detected pacing pulse from the CRM device. Furthermore, the baroreflex activation device may include a pulse generator for providing baroreflex stimulation pulse. The adjusting of the BAT may include changing one or more characteristics of pulses generated by the baroreflex activation device, as for example, generated by the pulse generator. The characteristics of the pulse generated by the baroreflex activation device includes, by way of example, any one or more of pulse amplitude, frequency, burst energy, and wave characteristics.

The pacing pulse generated by the CRM device is typically responsive to at least one or more of heart rate, cardiac waveform, timing of arterial and/or ventricular contractions, venous or arterial pressure, venous or arterial volume, cardiac output, pressure and/or volume in one or more heart chambers, cardiac efficiency, cardiac impedance, and edema.

In an exemplary embodiment, the present method, may be used to treat any number of conditions, such as hypertension, as well as resynchronization of the patient's heart. Additionally, or alternatively, the present method may be used to treat hypertension cardiac arrhythmia.

In another exemplary method for treating a patient according to the present invention, the method, it might be beneficial to assess during which of the cardiac phases the BAT may be more effective. In determining the more responsive cardiac phase, an exemplary method includes, stimulating a baroreflex system of the patient with a baroreflex activation device according to a baroreflex activation therapy (“BAT”) during systole and diastole cardiac phases, and determining the patient's response to the baroreflex stimulation during the systole and diastole cardiac phases. Thereafter, it is determined during which of the cardiac phases the BAT provides better therapy to the patient. A pre-determined time delay may be selected between the BAT such that the therapy is delivered during the preferred cardiac phase of systole or diastole. Furthermore, a pacing pulse generated from a CRM device is detected by the baroreflex activation device. Such detection, may be achieved by any suitable means, such as the baroreflex activation device being connectable to an output terminal of the CRM device. The baroreflex system therapy may be thereafter adjusted in response to the detected pacing pulse. The baroreflex therapy may be delivered after the pre-determined time delay. The adjusting of the of the BAT may be performed in a continuous loop, as necessary, to maintain effective BAT.

In another exemplary method for treating a patient according to the present invention, a baroreflex system of a patient is stimulated according to a BAT during the systole and the diastole of the cardiac phases; and measuring the patient's response to the stimulation during the systole and diastole cardiac phases is measured, and thereafter it is determined which of the systole and diastole cardiac phases is more responsive to the BAT and is the preferred cardiac phase for baroreflex stimulation. The BAT may be delivered after a pre-determined time delay such that the BAT is delivered during the preferred cardiac phase of systole or diastole. The method may further include the stimulation of the baroreflex system of the patient according to a therapy during the preferred cardiac phase. The baroreflex activation device is connectable to an output terminal of the CRM device, the method further comprising continuously detecting the pacing pulse. The BAT may be adjusted in response to the detected pacing pulse from the CRM device.

An exemplary embodiment of a system for treating a patient, includes a baroreflex activation device connected to an output terminal of a pacing pulse generator of a CRM device. The baroreflex activation device is configured to stimulate the baroreflex system of the patient according to a baroreflex activation therapy which is deliverable by the baroreflex activation device. A pre-determined time delay period prior to delivery of the BAT is established, during which the pacing pulse activity generated by the CRM device is monitored. Once the time delay has expired and a pacing pulse is detected, BAT is delivered to the patient. In some embodiments, such BAT is delivered even if no pacing pulse is detected after expiration of the controlled time period. In some embodiments, the CRM is continuously monitored for generation of pacing pulse, until at least such time that the baroreflex activation device detects a pacing pulse. The time delay may be based on the determination of which of the systole or diastole cardiac phases is more responsive to the BAT.

In another exemplary embodiment of a method for treating a patient, a baroreflex activation device connectable to an output terminal of a CRM device for generation of pacing pulses is provided along with means for actively detecting the pacing pulse generated by the CRM device.

The baroreflex activation device may be programmable to apply a BAT which includes a plurality of therapy regimens. The CRM device may include any one or more of a cardiac pacemaker, an implantable defibrillator, or a combination of such devices, such as an integrated pacemaker/defibrillator.

In another exemplary embodiment of a method for treating a patient, a contraction in a heart of the patient is detected and a baroreflex system of the patient is activated after expiration of a pre-selected period of time. The contraction may include the detection of electrical activity in the heart, as for example, detecting an R-wave in an electrocardiogram signal from the heart of the patient. Additionally, or alternatively, the detection of the contraction is achieved by way of detecting an electric pulse generated by a CRM device.

In another exemplary embodiment of a method for treating a patient, an electric pulse generated by a CRM device is detected and a baroreflex system of the patient after expiration of a pre-selected period of time is activated. The CRM device, as indicated above, may be a pacemaker, a cardiac resynchronization therapy (CRT) device, or a cardioverter. The electric pulse generated by the CRM device is detected by measuring a voltage difference between a BAT electrode and a housing of a BAT device. The electric pulse generated by the CRM device may be detected measuring a voltage difference between a lead of the CRM device and the housing of the BAT device. Similarly, the electric pulse generated by the CRM device may be detected by measuring a voltage difference between a lead of the CRM device and an electrode of a BAT device.

In another exemplary embodiment of a method for treating a patient, a preferred phase of the patient's cardiac cycle for delivery of BAT to the patient's baroreflex system is identified and the baroreflex system of the patient is activated during the preferred phase of the patient's cardiac cycle. The preferred cardiac phase may be identified by stimulating the baroreflex system of the patient during both the systolic and diastolic phases of the patient's cardiac cycle and quantifying the responses based on the patient's response to the baroreflex stimulation during these phases. The two responses are thereafter compared and the more responsive cardiac cycle phase is identified, and used for the delivery of the BAT.

In another exemplary embodiment of a method for treating a patient, the ventricular fibrillation in a heart of the patient detected and the heart is defibrillated. The baroreflex system of the patient is activated to reduce the likelihood of the occurrence of ventricular fibrillation.

In another exemplary embodiment of a method for treating a patient, an electric pulse generated by a CRM device is detected and a baroreflex system of the patient is activated in response to the detected electric pulse. The CRM device, as indicated above, may be a defibrillator. The activation of the baroreflex system of the patient may be discontinued after expiration of a pre-selected period of time.

In another exemplary embodiment of a method for treating a patient, baroreflex activation therapy to a body of the patient is delivered and an electrical pulse generated by a CRM device is detected, which may be an implantable defibrillator. The intensity of the baroreflex activation therapy is increased upon detection of the electrical pulse. The baroreflex activation therapy may be returned to an original intensity after expiration of a pre-selected period of time.

In another exemplary embodiment of a method for treating a patient, baroreflex activation therapy is delivered to a body of the patient at a first intensity, optionally, after expiration of a pre-selected period of time, and an electrical pulse generated by a CRM device is detected. The baroreflex activation therapy is delivered to the body of the patient at a second intensity, which may be greater than the first intensity, after the electrical pulse has been detected. As mentioned earlier, the CRM device may be an implantable defibrillator.

In an exemplary embodiment of a baroreflex activation system for treating a patient, a system, according to the present invention, includes a baroreflex activation device which is connectable to an output terminal of a CRM device for generation of pacing pulses. Furthermore, the system includes means for actively detecting pacing pulses generated by the CRM device. The baroreflex activation device may be programmable to apply a BAT including a plurality of therapy regimens. The CRM device may include any one or more of a cardiac pacemaker, an implantable defibrillator, or a combination device thereof.

Optionally, any system according to the present invention, may also include at least one sensor connectable with the CRM device for sensing one or more patient conditions. Such a system may further include a processor connectable with the sensor for processing the sensed patient condition(s) into data and providing the data to the CRM device. The sensor may be any one or more of suitable physiological sensors such as an electrocardiogram, a pressure sensing device, a volume sensing device, an accelerometer, or an edema sensor. In various embodiments, sensor(s) may be adapted to sense heart rate, cardiac waveform, timing of atrial and/or ventricular contractions, venous or arterial pressure, venous or arterial volume, cardiac output, pressure and/or volume in one or more heart chambers, cardiac efficiency, cardiac impedance, edema and/or the like.

Generally, any of a number of suitable anatomical structures may be activated to provide baroreflex activation. For example, in various embodiments, activating the baroreflex system may involve activating one or more baroreceptors, one or more nerves coupled with a baroreceptor, a carotid sinus nerve, or some combination thereof. In embodiments where one or more baroreceptors are activated, the baroreceptor(s) may sometimes be located in arterial vasculature, such as but not limited to a carotid sinus, aortic arch, heart, common carotid artery, subclavian artery, pulmonary artery, femoral artery and/or brachiocephalic artery. Alternatively, a baroreflex activation device may be positioned in the low-pressure side of the heart or vasculature, as described in U.S. patent application Ser. No. 10/284,063, previously incorporated by reference, in locations such as an inferior vena cava, superior vena cava, portal vein, jugular vein, subclavian vein, iliac vein, azygous vein, pulmonary vein and/or femoral vein. In many embodiments, the baroreflex activation device is implanted in the patient. The baroreflex activation may be achieved, in various embodiments, by electrical activation, mechanical activation, thermal activation and/or chemical activation. Furthermore, baroreflex activation may be continuous, pulsed, periodic or some combination thereof in various embodiments.

It should further be appreciated by those skilled in the art that although the present invention may be discussed and is of particular relevance to baroreflex activation device with implantable pulse generators, it is also applicable to baroreflex activation devices with external pulse generators. Thus, the present invention and all embodiments described herein are applicable to baroreflex activation devices with pulse generators which are external (and not implantable) as well as those which are implantable. It should be further understood by those skilled in the art that the methods, devices, and systems according to the present invention are further applicable to modifying any one or more of the nervous system activity of the patient, autonomic nervous system activity of the patient, sympathetic/parasympathetic nervous system activity of the patient, or metabolic activity of the patient.

### BRIEF DESCRIPTION OF THE DRAWINGS

FIG. 1 is a schematic illustration of the upper torso of a human body showing the major arteries and veins and associated anatomy.

FIG. 2A is a cross sectional schematic illustration of a carotid sinus and baroreceptors within a vascular wall.

FIG. 2B is a schematic illustration of baroreceptors within a vascular wall and the baroreflex system.

FIG. 3 is a schematic view of a system illustrating features of an exemplary embodiment of the present invention.

FIG. 4A is a schematic view of an exemplary pacemaker and a baroreflex activation therapy device implanted in a body of a patient including features of the present invention.

FIG. 4B is a schematic view illustrating a cardioverter and a BAT device implanted in a body of a patient and including features of the present invention.

FIG. 4C is a schematic view illustrating a cardiac rhythm management device and a BAT device implanted in a body of a patient.

FIG. 5A is a block diagram illustrating an exemplary patient treatment system embodying features of the present invention.

FIG. 5B is a block diagram illustrating another exemplary patient treatment system embodying features of the present invention.

FIG. 6, is a block diagram illustrating an exemplary system including a baroreflex activation therapy device in communication with a cardiac rhythm management device.

FIG. 7, in a block diagram of illustrating an exemplary method for evaluation of the response of a patient to baroreflex therapy during systole and diastole cardiac cycles.

FIG. 8A is a simplified flow chart indicative of an exemplary process with the electronics of a baroreflex activation device coupled to the output terminal of a cardiac rhythm management device, in accordance with the present invention.

FIG. 8B is a simplified flow chart indicative of another exemplary process with the electronics of a baroreflex activation device coupled to the output terminal of a cardiac rhythm management device, in accordance with the present invention.

FIG. 8C is a simplified flow chart indicative of another exemplary process with the electronics of a baroreflex activation device coupled to the output terminal of a cardiac rhythm management device, in accordance with the present invention.

FIG. 9 is an exemplary illustration of pacing pulses generated by a cardiac rhythm management device and the signal for enabling (starting) the baroreflex therapy.

FIG. 10 is a timing diagram illustrating an arterial pressure waveform and a logical baroreflex activation therapy enabled signal.

FIG. 11 is a graphical representation of baroreflex activation therapy intensity as a function of heart rate.

### DETAILED DESCRIPTION OF THE INVENTION

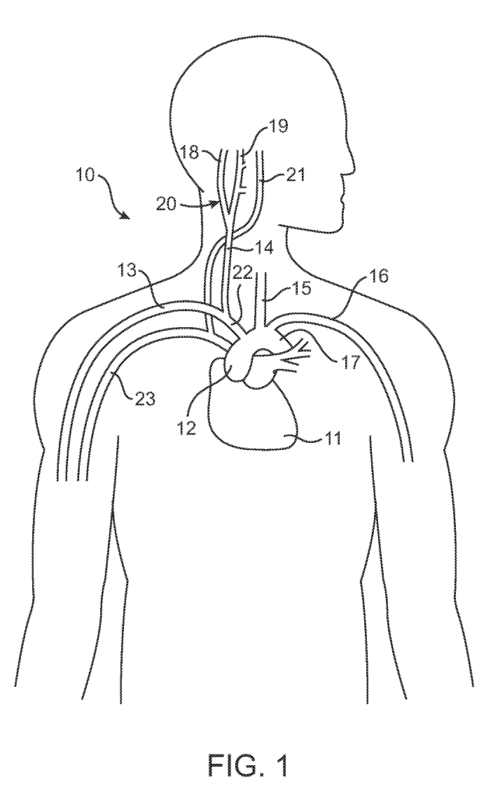

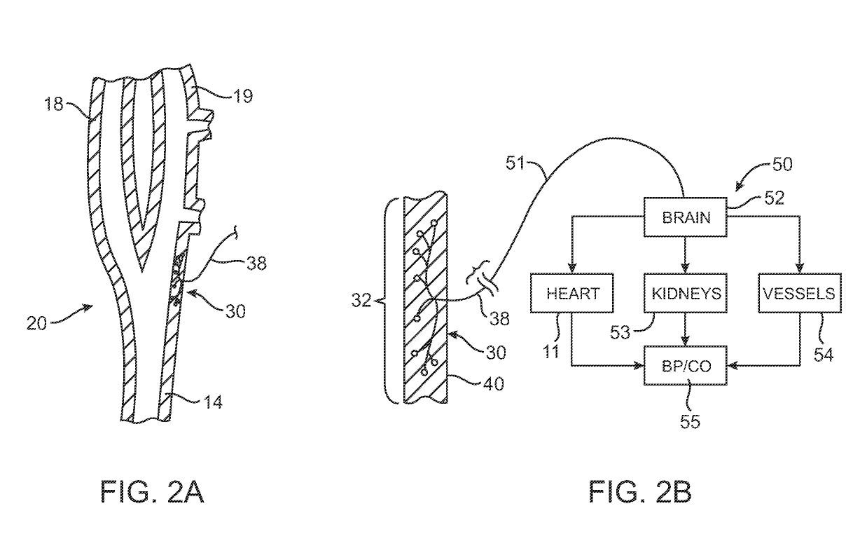

Referring now to FIGS. 1, 2A, and 2B, within the arterial walls of the aortic arch 12, common carotid arteries 14/15 (near the right carotid sinus 20 and left carotid sinus), subclavian arteries 13/16, and brachiocephalic artery 22, baroreceptors 30 are shown. For example, as best seen in FIG. 2A, baroreceptors 30 reside within the vascular walls of the carotid sinus 20. Baroreceptors 30 are a type of stretch receptor used by the body to sense blood pressure. An increase in blood pressure causes the arterial wall to stretch, and a decrease in blood pressure causes the arterial wall to return to its original size. Such a cycle is repeated with each beat of the heart. Baroreceptors 30 located in the right carotid sinus 20, the left carotid sinus, and the aortic arch 12 play the most significant role in sensing blood pressure that affects baroreflex system 50, which is described in more detail with reference to FIG. 2B.

With reference now to FIG. 2B, a schematic illustration shows baroreceptors 30 disposed in a generic vascular wall 40 and a schematic flow chart of baroreflex system 50. Baroreceptors 30 are profusely distributed within the arterial walls 40 of the major arteries discussed previously, and generally form an arbor 32. The baroreceptor arbor 32 comprises a plurality of baroreceptors 30, each of which transmits baroreceptor signals to the brain 52 via nerve 38. Baroreceptors 30 are so profusely distributed and arborized within the vascular wall 40 that discrete baroreceptor arbors 32 are not readily discernable. To this end, baroreceptors 30 shown in FIG. 2B are primarily schematic for purposes of illustration.

In addition to baroreceptors, other nervous system tissues are capable of inducing baroreflex activation. For example, baroreflex activation may be achieved in various embodiments by activating one or more baroreceptors, one or more nerves coupled with one or more baroreceptors, a carotid sinus nerve, or some combination thereof. Therefore, the phrase “baroreflex activation” generally refers to activation of the baroreflex system by any means, and is not limited to directly activating baroreceptor(s). Although the following description often focuses on baroreflex activation/stimulation and induction of baroreceptor signals, various embodiments of the present invention may alternatively achieve baroreflex activation by activating any other suitable tissue or structure. Thus, the terms “baroreflex activation device” and “baroreflex stimulation device” are used interchangeably in this application.

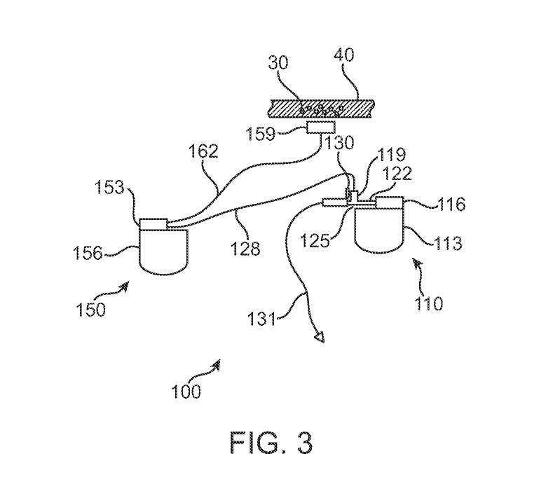

FIG. 3 is a schematic view of a system 100 in accordance with an exemplary embodiment, including a baroreflex activation therapy (“BAT”) device 150 and a CRM pacemaker 110 that are shown in relationship with one another. As shown, the baroreflex activation device and the CRM device, are electrically connected to one another through lead 128.

In the exemplary embodiment shown in FIG. 3, pacemaker 110 includes a pacemaker housing 113 and a pacemaker header 116. In FIG. 3, a T-shaped connector 119 is shown extending from pacemaker header 116. T-shaped connector 119 includes a male connection 122, a female connection 125, and a lead wire 128. In the embodiment of FIG. 3, male connection 122, female connection 125, and lead wire 128 are all electrically connected to one another.

In some useful embodiments, male connection 122 is dimensioned to be inserted into a pacemaker port that is designed to receive a pacemaker lead 131. For example, male connection 122 may be dimensioned in accordance with pacemaker lead standards developed by the International Standards Organization (ISO). One example of such a standard is ISO 5841-3. A port in the header of a pacemaker which conforms to this standard may be referred to as an IS-1 port.

In some other useful embodiments, female connection 125 comprises an IS-1 port. A pacing lead 131 including a male connector 122 is shown in FIG. 3. In the embodiment of FIG. 3, male connector 130 of pacing lead 131 has been inserted into female connection 125 of T-shaped connector 119.

In the exemplary embodiment shown in FIG. 3, lead wire 128 of T-shaped connector 119 is connected to a BAT header 153 of BAT device 150. Accordingly, lead wire 128 creates an electrical connection between pacing lead 131 and BAT device 150. In some useful embodiments, this electrical connection allows BAT device 150 to measure the electrical potential difference between pacing lead 131 and a BAT housing 156 of BAT device 150. In these useful embodiments, measuring this potential difference may allow BAT device 150 to detect pacing pulses delivered to pacing lead 131 by pacemaker 110.

A BAT electrode assembly 159 is also shown in FIG. 3. BAT electrode assembly 159 is electrically connected to BAT device 150 by a BAT lead wire 162. With reference to FIG. 3, it will be appreciated that BAT electrode assembly 159 is located near a number of baroreceptors 30 located in a blood vessel wall. In some useful embodiments of the present invention, BAT device 150 may activate a baroreflex system of a patient by causing electrical current to flow between two or more electrodes of BAT electrodes assembly 150. Also in some useful embodiments, BAT device 150 may measure the electrical potential difference between pacing lead 131 and a BAT electrode assembly 159. In these useful embodiments, measuring this potential difference may allow BAT device 150 to detect pacing pulses delivered to pacing lead 131 by pacemaker 110.

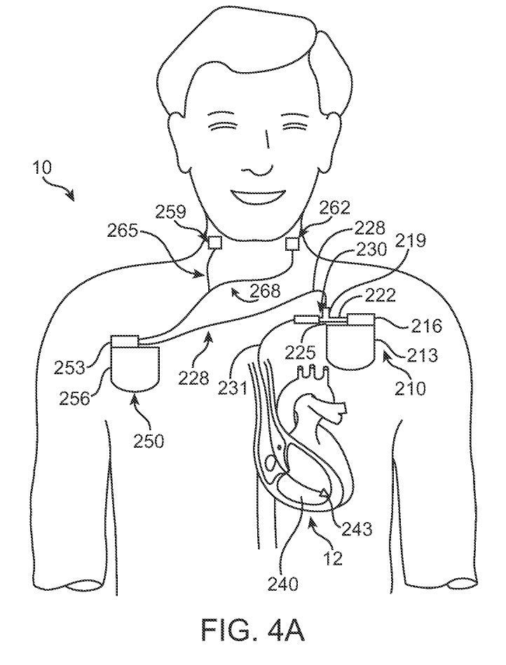

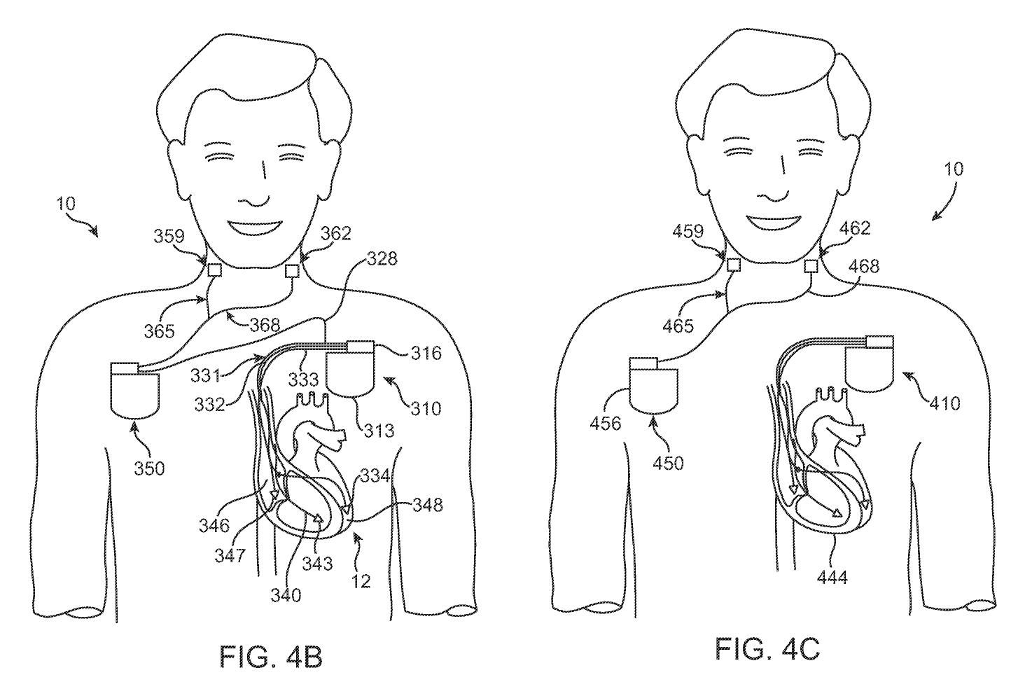

FIG. 4A is a schematic view showing a pacemaker 210 and a BAT device 250 that have been implanted in a body of a patient 10. In the exemplary embodiment of FIG. 4A, pacemaker 210 includes a pacemaker housing 213 and a pacemaker header 216. A pacing lead 231 is connected to a port of pacemaker header 216 via a T-shaped connector 219. T-shaped connector 219 includes a male connection 222, a female connection 225, and a lead wire 228. In the embodiment of FIG. 4A, male connection 222, female connection 225, and lead wire 228 are all electrically connected to one another. A connector 230 of pacing lead 231 forms an electrical connection with female connection 225 of T-shaped connector 219. Male connection 222 forms an electrical connection with a port of pacemaker header 216.

In some useful embodiments, male connection 222 and female connection 225 are both dimensioned in accordance with ISO 5841-3. In these useful embodiments, T-shaped connector 219 can be interposed between a pacing lead having an IS-1 connector and an pacemaker having an IS-1 port.

In FIG. 4A, pacing lead 231 is shown extending into a right ventricle 240 of a heart 12 of patient 10. Pacing lead 231 includes a pacing electrode 243 that is represented by a triangle in FIG. 4A. In the embodiment of FIG. 4A, pacemaker 210 may deliver pacing pulses to heart 12 via pacing lead 231 and pacing electrode 243. Also in the embodiment of FIG. 4A, BAT device 250 may detect these pacing pulses via a lead wire 228 of T-shaped connector 219.

In the embodiment of FIG. 4A, lead wire 228 of T-shaped connector 219 is connected to a BAT header 253 of BAT device 250. Accordingly, lead wire 228 creates an electrical connection between pacing lead 231 and BAT device 250. In some useful embodiments, this electrical connection allows BAT device 250 to measure the electrical potential between pacing lead 231 and a BAT housing 256 of BAT device 250. In these useful embodiments, measuring this potential difference may allow BAT device 250 to detect pacing pulses delivered to pacing lead 231 by pacemaker 210. A first BAT electrode assembly 259 and a second BAT electrode assembly 262 are shown in FIG. 4A. Each BAT electrode assembly may comprise one or more BAT electrodes. First BAT electrode assembly 259 and second BAT electrode assembly 262 are electrically connected to BAT device 250 by BAT lead wires 265 and 268, respectively. In some useful embodiments of the present invention, BAT device 250 may activate a baroreflex system of patient 10 by causing electrical current to flow between two or more electrodes of first BAT electrode assembly 259 and/or second BAT electrode assembly 262. Also in some useful embodiments, BAT device 250 may measure the electrical potential difference between pacing lead 231 and one or more of the BAT electrode assemblies 259, 262. In these useful embodiments, measuring this potential difference may allow BAT device 250 to detect pacing pulses delivered to pacing lead 231 by pacemaker 210.

FIG. 4B is a schematic view showing a cardioverter 310 and a BAT device 350 that have been implanted in a body of a patient 10. In the embodiment of FIG. 4B, cardioverter 310 includes a cardioverter housing 313 and a cardioverter header 316. A first cardiac lead 331, a second cardiac lead 332, and a third cardiac lead 333 are each connected to a port of cardioverter header 316.

In FIG. 4B, first cardiac lead 331 is shown extending into a right ventricle 340 of a heart 12 of patient 10. First cardiac lead 331 includes a pacing electrode 343 that is represented by a triangle in FIG. 4B. Cardioverter 310 may deliver pacing pulses to right ventricle 340 of heart 12 via first cardiac lead 331. In the embodiment of FIG. 4B, BAT device 350 is electrically connected to first cardiac lead 331 by a lead wire 328. Accordingly, BAT device 350 is capable of detecting pacing pulses delivered to right ventricle 340.

In FIG. 4B, second cardiac lead 332 is shown extending into a right atrium 346 of the heart 12 of patient 10. Second cardiac lead 332 includes a pacing electrode 347 that is represented by a triangle in FIG. 4B. Accordingly, cardioverter 310 may deliver pacing pulses to right atrium 346 of heart 12 using second cardiac lead 332. In the embodiment of FIG. 4B, third cardiac lead 333 includes a pacing electrode 334 that is located proximate a left ventricle 348 of heart 12. Using third cardiac lead, cardioverter 310 may deliver pacing pulses to left ventricle 334 of heart 12.

A first BAT electrode assembly 359 and a second BAT electrode assembly 362 are shown in FIG. 4B. Each BAT electrode assembly may comprise one or more BAT electrodes. First BAT electrode assembly 359 and second BAT electrode assembly 362 are electrically connected to BAT device 350 by BAT lead wires 365 and 368, respectively. In some useful embodiments of the present invention, BAT device 350 may activate a baroreflex system of patient 10 by causing electrical current to flow between two or more electrodes of first BAT electrode assembly 359 and/or second BAT electrode assembly 362.

FIG. 4C is a schematic view showing a CRM device 410 and a BAT device 450 that have been implanted in a body of a patient 10. In the exemplary embodiment of FIG. 4C, CRM device 410 may comprise, for example, a cardiac pacemaker, an implantable defibrillator, a cardioverter, a cardiac resynchronization device, or the like. A first BAT electrode assembly 459 and second BAT electrode assembly 462 are electrically connected to BAT device 450 by BAT lead wires 465 and 468, respectively. Each BAT electrode assembly may comprise one or more BAT electrodes. BAT device 450 may activate a baroreflex system of patient 10 by causing electrical current to flow between two or more electrodes of first BAT electrode assembly 459 and/or second BAT electrode assembly 462.

In some useful embodiments, BAT device 450 may measure the electrical potential differences between different locations within the body of patient 10. For example, BAT device 450 may measure voltage differences between first BAT electrode assembly 459 and BAT housing 456 of BAT device 450. BAT device 450 may also measure voltage differences between second BAT electrode assembly 462 and BAT housing 456 of a BAT device 450. Additionally, BAT device 450 may measure voltage differences between first BAT electrode assembly 459 and second BAT electrode assembly 462. These measurements may be used, for example, to monitor electrical activity in a heart 444 of patient 10. By way of a second example, these measurements may be used to detect electrical pulses produced by CRM device 410.

As mentioned above, BAT device 450 may measure electrical potential differences between different locations within the body of patient 10 for monitoring electrical activity in heart 444 of patient 10. When this is the case, BAT device 450 may repeatedly measure and record at least one electrical potential difference to obtain a digitized electrocardiogram waveform. By analyzing these measured values and/or the electrocardiogram waveform, BAT device 450 can detect a contraction in heart 444 of patient 10. In some embodiments, for example, BAT device 450 may detect a contraction in heart 444 by identifying an R-wave component of the electrocardiogram waveform. After detecting a contraction in heart 444 of patient 10, BAT device 450 may use that information to adjust the timing and/or intensity of BAT therapy pulses.

As mentioned above, BAT device 450 may measure electrical potential differences between different locations within the body of patient 10 to detect electrical pulses produced by CRM device 410. If CRM device 410 is a pacemaker, for example, BAT device 450 may detect pacing pulses produced by CRM device 410 by periodically measuring electrical potential differences within the body of patient 10. After detecting one or more pacing pulses, BAT device 450 may use this information to adjust the timing and/or intensity of BAT therapy pulses. If CRM device 410 is a defibrillator, BAT device 450 may detect a one or more defibrillation pulses produced by CRM device 410. An implantable defibrillator produces a defibrillation pulse when it has determined that the patient is suffering from ventricular fibrillation. The defibrillation pulse is an electrical shock waveform designed to terminate the ventricular fibrillation and facilitate the heart's return to a normal rhythm. For a period of time immediately following an incident of ventricular fibrillation, there may be an elevated risk that ventricular fibrillation will reoccur. Delivering BAT to the patient for a period of time after defibrillation may reduce the likelihood that ventricular fibrillation will reoccur. In some implementations, the patient may have be receiving BAT at the time defibrillation occurs. When this is the case, BAT device 450 may respond to the detection of a defibrillation pulse by delivering BAT at a greater intensity for a period of time after defibrillation has occurred. This may reduce the likelihood that ventricular fibrillation will reoccur, for example, by reducing the patient's blood pressure. By way of a second example, this may reduce the likelihood that ventricular fibrillation will reoccur, by reducing the physiologic demand placed on the heart (e.g., the heart will be required to pump less blood).

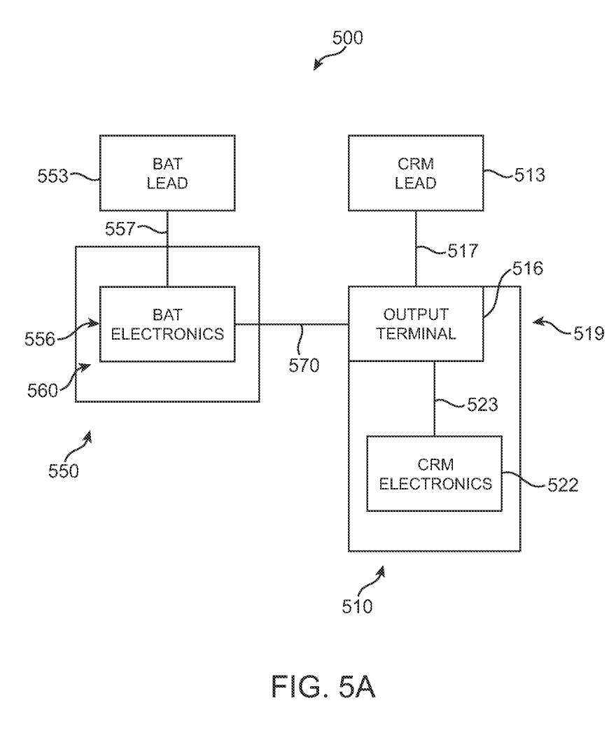

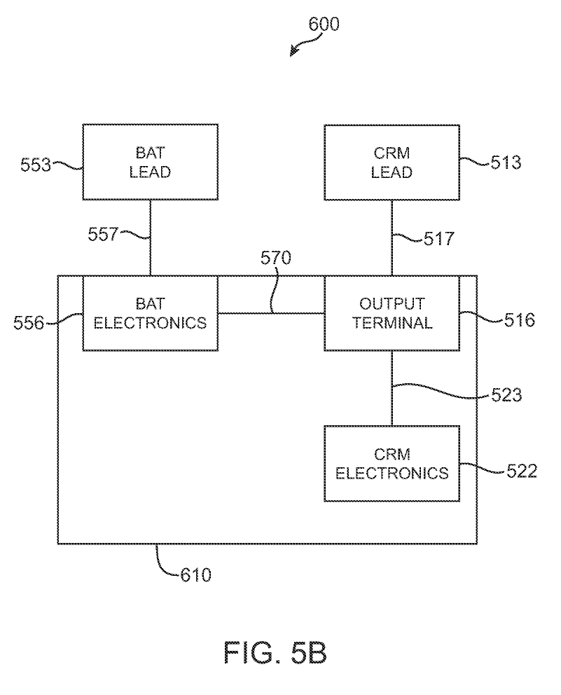

FIG. 5A is a block diagram illustrating an exemplary patient treatment system 500. System 500 comprises a baroreflex activation therapy (BAT) device 550 and cardiac rhythm management (CRM) device 510. BAT device 550 comprises a BAT lead 553 that is electrically connected to BAT electronics 556 via connection 557. With reference to FIG. 5A, it will be appreciated that BAT electronics 556 is disposed in a cavity defined by a BAT housing 560. BAT electronics 556 is capable of producing baroreflex activation therapy pulses that are delivered to a patient via a BAT lead 553.

CRM device 510 comprises a CRM lead 513 that is electrically connected to an output terminal 516 of CRM device 510 via connection 517. CRM device 510 also comprises a CRM housing 519 and CRM electronics 522. As shown in FIG. 5A, CRM electronics 522 is electrically connected to output terminal 516 via connection 523, and is located inside CRM housing 519. CRM electronics 522 is capable of producing CRM pulses (e.g., pacing pulses, cardiac resynchronization pulses, and defibrillation pulses) that are delivered to a patient via a CRM lead 513.

In the embodiment of FIG. 5A, BAT electronics 556 is electrically connected to output terminal 516 of CRM device 510 by a lead wire 570. In some useful embodiments, this electrical connection allows BAT device 550 to measure the electrical potential between CRM lead 513 and a BAT housing of BAT device 560. In these useful embodiments, measuring this potential difference may allow BAT device 550 to detect electrical pulses delivered to CRM lead 513 by CRM electronics 522.

FIG. 5B illustrates another embodiment of the system 500 shown in FIG. 5A, except that the baroreflex activation device and the CRM device are housed together in a single housing 610.

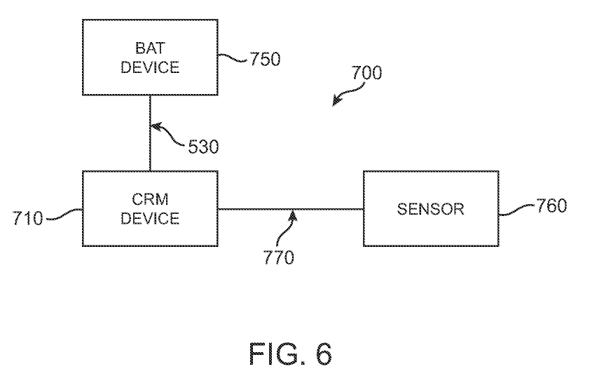

Now referring to FIG. 6, an exemplary system 700 is shown including a BAT device 750 that is electrically connected to a CRM device 710 by a lead 530. The CRM device 710 is connected to a sensor 760 by way of a leads 770.

In various alternative embodiments, the sensor 760 (or multiple sensors) may be coupled with the CRM device 710. In an alternative embodiment, the baroreflex activation device 750 and the CRM device 710 may be combined into on unitary device, with the unitary device being coupled with one or more sensors. In yet another embodiment, the unitary device may also be combined with one or more built-in sensors 760. In some embodiments, the sensor 760 (or multiple sensors) may be coupled directly with either or both the baroreflex activation device and the CRM device.

CRM devices 710 are known in the art, and any suitable CRM device 710 now known or hereafter developed may be used in various embodiments of the present invention. For example, the CRM device 114 may be the same as or similar to those described in U.S. Pat. Nos. 6,768,923; 6,766,189; 6,748,272; 6,704,598; 6,701,186; and 6,666,826, which were previously incorporated by reference. Alternatively, any other suitable CRM device may be incorporated into the heart failure treatment system. In some embodiments, CRM device 710 may comprise a combined pacemaker/defibrillator, and in some cases, a biventricular pacemaker/defibrillator.

Any suitable baroreflex activation device 750 (or multiple devices) may also be used, in various embodiments. Examples of suitable baroreflex activation devices 112 include, but are not limited to, those described in detail in U.S. Pat. Nos. 6,522,926 and 6,616,624, and U.S. patent application Ser. Nos. 09/964,079, 09/963,777, 09/963,991, 10/284,063, 10/453,678, 10/402,911, 10/402,393, 10/818,738, and 60/584,730, which were previously incorporated by reference. Any number or type of suitable baroreflex activation device may be used, in accordance with various embodiments, and the activation device(s) may be placed in any suitable anatomical location. For further details regarding specific exemplary baroreflex activation devices, reference may be made to any of the patents or patent applications listed immediately above.

The sensor 760 (or in some embodiments multiple sensors) may include any suitable sensor device or combination of devices. Oftentimes, the sensor(s) is adapted for positioning in or on the heart, although in various alternative embodiments sensor(s) may be placed in one or more blood vessels, subcutaneously, in any other suitable location in the patient, or even outside the patient, such as with an external electrocardiogram device. Examples of sensors include, but are not limited to, electrocardiogram devices, pressure sensors, volume sensors, accelerometers, edema sensors and/or the like. Sensor(s) may sense any suitable patient characteristic (or condition), such as but not limited to heart rate, cardiac waveform, timing of atrial and/or ventricular contractions, venous or arterial pressure, venous or arterial volume, cardiac output, pressure and/or volume in one or more heart chambers, cardiac efficiency, cardiac impedance and/or edema. Again, in various embodiments any suitable sensor device(s) may be used and any suitable condition may be sensed.

Generally, the sensor 760 may provide information about sensed patient conditions either to the baroreflex activation device or the CRM device. In some embodiments, such information may then be used by either or both the baroreflex activation device 750 and the CRM device 710 to either initiate or modify a treatment. Typically, though not necessarily, the system 700 includes a processor for converting sensed information into data that is usable by either or both CRM and the baroreflex activation device. Normally, the sensor is configured for sensing a physiological cardiac response, transmitting to the CRM device which in turn may affect the pacing pulse generated by the CRM device. The baroreflex activation device, which is connected to the output terminal of the CRM device, may thereafter adjust its performance as may be needed.

The sensor 760 may comprise any suitable device that measures or monitors a parameter indicative of the need to modify or cardiac rhythm treatment. For example, the sensor may comprise a physiologic transducer or gauge that measures cardiac activity, such as an ECG. Alternatively, the sensor may measure cardiac activity by any other technique, such as by measuring changes in intracardiac pressures or the like. Examples of suitable transducers or gauges for the sensor include ECG electrodes and the like. Although only one sensor is shown, multiple sensors of the same or different type at the same or different locations may be utilized. The sensor is preferably positioned on or near the patient's heart, on or near major vascular structures such as the thoracic aorta, or in another suitable location to measure cardiac activity, such as increased heart rate or pressure changes. The sensor may be disposed either inside or outside the body in various embodiments, depending on the type of transducer or gauge utilized.

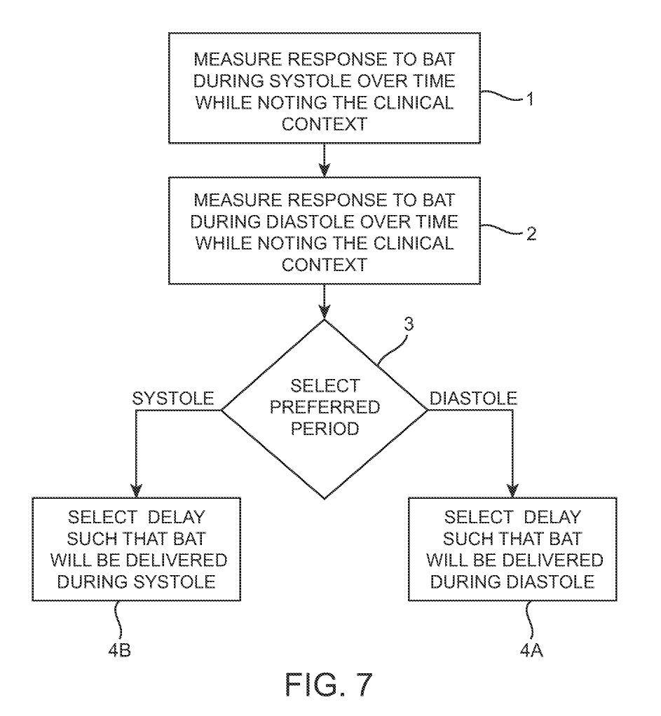

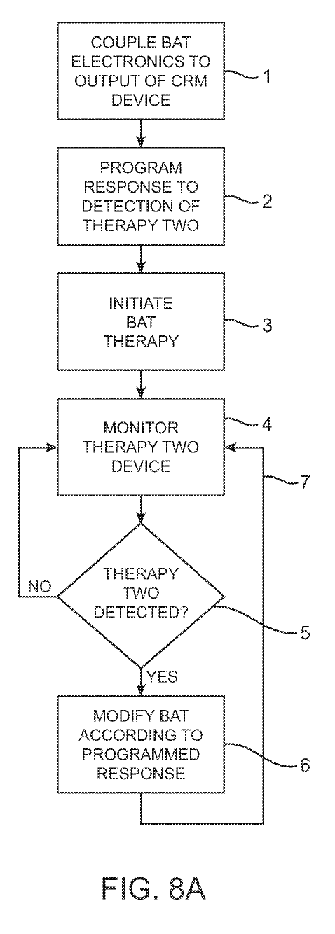

Now referring to FIG. 7, in an exemplary method, the response of a patient to baroreflex therapy is measured during systolic and diastolic cardiac phases (boxes 1 & 2) while noting the clinical context in which the measurements are made. It is, thereafter, determined during which of the cardiac phases (systole or diastole) the patient's response to baroreflex therapy is more preferred (box 3). A time delay is selected such that the baroreflex therapy is delivered to the patient during the preferred cardiac phase of diastole or systole (boxes 4A and 4B).

Measuring the response of the patient to baroreflex activation therapy during systole and diastole may include measuring a patient parameter under three different conditions. First, when no baroreflex activation therapy pulses are being delivered to the patient. Second, when baroreflex activation therapy pulses are being delivered to the patient during the systolic phase of the cardiac cycle. Third, when baroreflex activation therapy pulses are being delivered to the patient during the diasystolic phase of the cardiac cycle. The therapeutic effect of delivering baroreflex activation therapy pulses during the systolic phase of the cardiac cycle can be determined by subtracting the first measurement from the second measurement. The therapeutic effect of delivering baroreflex activation therapy pulses during the diasystolic phase of the cardiac cycle can be determined by subtracting the first measurement from the third measurement.

Various patient parameters can be measured during the performance of the method illustrated in FIG. 7. Examples of patient parameters that may be suitable in some applications include, but are not limited to: blood pressure, oxygen saturation, heart rate and electrocardiogram waveform characteristics. Blood pressure may be measured, for example, by placing a pressure sensor in contact with the blood. Oxygen saturation may be measured, for example, using pulse oximetry techniques.

Heart rate may be determined, for example, by detecting contractions of the heart and calculating the frequency of those contractions. A BAT device in accordance with some exemplary embodiments may detect heart contractions by measuring electrical potential differences between different locations within the body of the patient. When this is the case, the BAT device may repeatedly measure and record electrical potential differences to obtain a digitized electrocardiogram waveform. By analyzing these measured values and/or the electrocardiogram waveform, the BAT device can identify an R-wave portion of each cardiac cycle. The BAT device may determine heart rate, for example, by measuring the time interval between the peak of one R-wave and the peak of another R-wave. Additional characteristics of the electrocardiogram waveform may also be measured and/or analyzed.

Determined during which of the cardiac phases (systole or diastole) the patient's response to baroreflex therapy is more preferred may include comparing the therapeutic effectiveness of baroreflex activation therapy pulses delivered during the systolic phase of the cardiac cycle with the therapeutic effectiveness of baroreflex activation therapy pulses delivered during the diasystolic phase of the cardiac cycle. For example, the change in a patient parameter that occurs when baroreflex activation therapy pulses are delivered during the systolic phase of the cardiac cycle may be mathematically compared with the change in a patient parameter that occurs when baroreflex activation therapy pulses delivered during the diasystolic phase of the cardiac cycle. This comparison may be used to identify the type of therapy that produces the largest desirable change in the patient parameter.

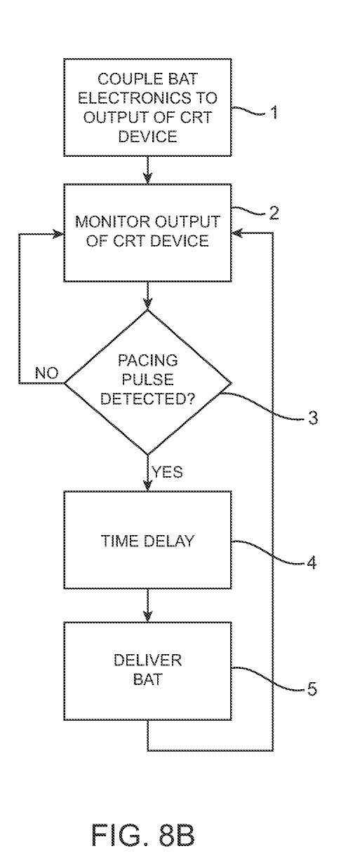

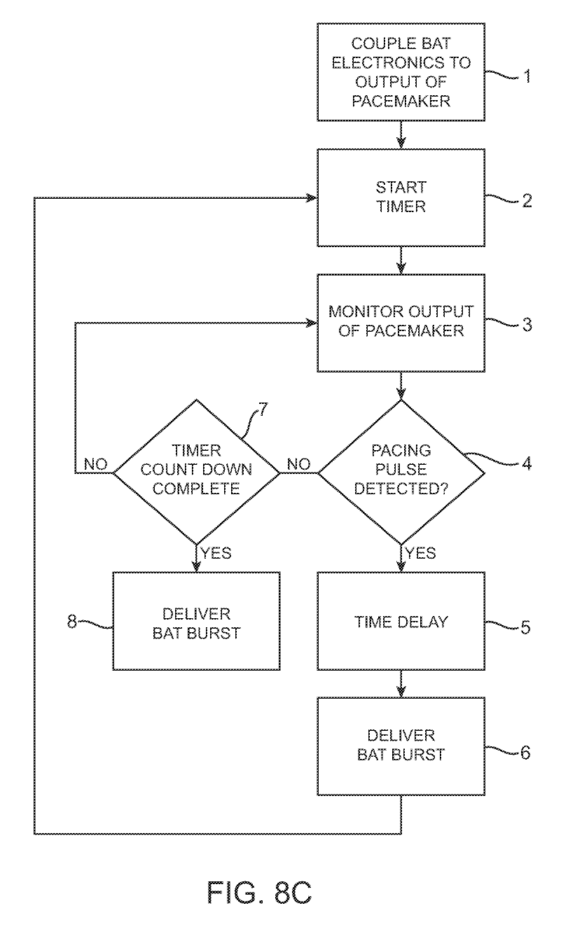

Now referring to FIG. 8A, a simplified flow chart indicative of an exemplary process is shown. Electronics of a baroreflex activation device is coupled to the output terminal of a CRM device (box 1). A healthcare provider (e.g., a physician) programs the BAT device with a response that the BAT device will undertake in the event that electrical pulses at the output terminal of the CRM device are detected (box 2). The baroreflex activation device initiates an appropriate baroreflex therapy (box 3). The output of the CRM device is monitored for any electrical pulses (box 4). If an electrical pulse is detected at the output terminal of the CRM device by the baroreflex activation device (box 5), the baroreflex activation device may modify the BAT therapy (box 6) that the patient is receiving in accordance with the pre-programmed response that was entered into the memory of the BAT device at box 2. If an electrical pulse from the CRM device is not detected, then the BAT device continues to deliver BAT in accordance with a prescribed therapy regimen. The output of the CRM device continues to be monitored (line 7) to comply with the necessary therapy regimen.

The exemplary method illustrated in FIG. 8A, may be used with various CRM devices. These CRM devices may include, for example, a cardiac pacemaker, an implantable defibrillator, a cardioverter, a cardiac resynchronization device, or the like. If the CRM device is a pacemaker, then the BAT device may, for example, detect pacing pulses produced by the pacemaker. After detecting one or more pacing pulses, the BAT device may use this information to adjust the timing and/or intensity of BAT therapy pulses. If the CRM device is a defibrillator, then the BAT device may detect a one or more defibrillation pulses produced by the defibrillator. The BAT device may respond to the detection of a defibrillation pulse by delivering BAT at a greater intensity for a period of time after defibrillation has occurred. This may reduce the likelihood that ventricular fibrillation will reoccur, for example, by reducing the patient's blood pressure. By way of a second example, this may reduce the likelihood that ventricular fibrillation will reoccur, by reducing the physiologic demand placed on the heart (e.g., the heart will be required to pump less blood).

Now referring to FIG. 8B, a simplified flow chart indicative of an exemplary process is illustrated. Electronics of a baroreflex activation device is electrically connected to an output terminal of a CRM device (box 1). The output of the CRM device is monitored by the baroreflex activation device (box 2). If a pacing pulse is detected (box 3) by the baroreflex activation device, the system awaits for a time delay (box 4) commensurate with a pre-selected time delay prior to the application of the baroreflex therapy (box 5). The magnitude of the time delay may be pre-selected, for example, in order to synchronize the delivery of baroreflex activation therapy with a preferred cardiac phase (e.g., systole or diastole). If at box 3, no pacing pulse is detected, then the baroreflex activation device continues to monitor the output of the CRM device until a pacing pulse is detected.

Now referring to FIG. 8C, a simplified flow chart of an exemplary process is illustrated. In some instances, it will be appropriate to deliver baroreflex activation therapy pulses even though the CRM device has not found it necessary to deliver a pacing pulse. In some exemplary methods, the timer is started at the end of each burst of baroreflex activation therapy. In these exemplary methods, the process ensures that the BAT electronics will continue to deliver baroreflex activation therapy to the patient even during periods when the patient does not require pacing. A burst of BAT will be delivered after a pre-selected period of time has passed, even if no pacing pulse has been detected.

As shown in FIG. 8C, the electronics of a baroreflex activation device is electrically connected to the output terminal of a CRM device which delivers pacing pulses (box 1). A timer is started (box 2). The output of the CRM device is monitored by the baroreflex activation device (box 3). If a pacing pulse produced by the CRM device is detected (box 4) the system awaits for a time delay (box 5) prior to the application of the baroreflex therapy (box 6). The magnitude of the time delay may be pre-selected, for example, in order to synchronize the delivery of baroreflex activation therapy with a preferred cardiac phase (e.g., systole or diastole). After the BAT burst is delivered, the process returns to box 2 with the timer restarted. If a pacing pulse (box 4) is not detected, the system determines whether the time set by the timer has expired (box 7). If the time has not expired, the system continues to monitor the output of the CRM device (box 3) for detection of a pacing pulse (box 4). If the time set by the timer (box 7) expires, then the baroreflex activation device delivers a burst of baroreflex therapy (box 8) and the timer is restarted (box 2).

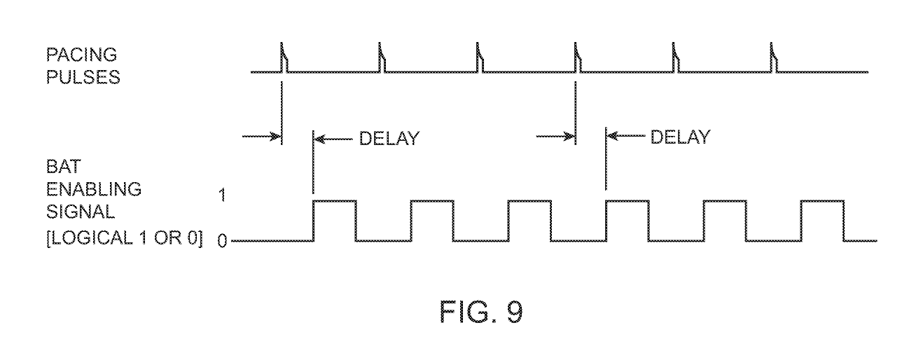

Now referring to FIG. 9, an exemplary illustration of pacing pulses generated by a CRM device and the signal for enabling (starting) the baroreflex therapy. The pacing pulses shown in FIG. 9 may be detected by a BAT device, for example, by connecting the BAT device to an output port of the CRM device that is producing the pacing pulses. By way of a second example, a BAT device may detect the pacing pulses by monitoring the voltage differential between two locations in the body of the patient. When this is the case, it is not necessary to form an electrical connection between the BAT device and the CRM device. Upon detection of the pacing pulses, the BAT device can use that information to select a desired timing for the delivery of BAT therapy pulses. As can be seen in FIG. 9, a time delay is applied between the pacing pulses detected by the baroreflex activation device and the delivery of the baroreflex therapy by the baroreflex activation device. The magnitude of the time delay may be pre-selected, for example, in order to synchronize the delivery of baroreflex activation therapy with a preferred cardiac phase (e.g., systole or diastole). The BAT device may infer that the patient's heart begins to contract when the pacing pulse is delivered. Each pacing pulse causes the left ventricle of the heart to contract and pump blood into the patient's arteries. Accordingly, a wave of relatively high pressure passes through the patient's arterial system after each contraction. Normally, this pressure wave can be expected to activate the baroreflex of the patient. Accordingly, delivering baroreflex therapy pulses slightly after the delivery of a pacing pulse may activate the baroreflex at a time when the body expects that baroreflex system to be activated. Timing baroreflex activation therapy in this way may achieve increased efficacy by mimicking the body's natural behavior.

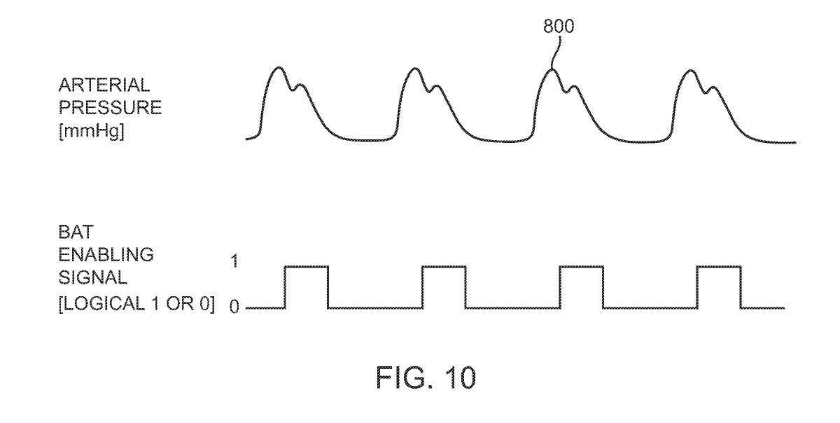

A desirable magnitude for the time delay illustrated in FIG. 9 may be determined, for example, by monitoring and analyzing electrical activity in the heart (e.g., the electrocardiogram waveform). The magnitude of the delay also may be determined, for example, using information gathered by analyzing an arterial pressure waveform that is collected by monitoring the output of a pressure sensor that is placed in contact with the blood. An exemplary arterial pressure waveform is illustrated in FIG. 10. The systolic and diastolic phases of the cardiac cycle are identifiable in both the electrocardiogram waveform and the arterial pressure waveform. Time intervals can also be measured in these waveforms for calculating a desired time delay.

FIG. 10 is a timing diagram illustrating an arterial pressure waveform 800 and a logical BAT enable signal. Arterial pressure waveform 800 of FIG. 10 includes a number of high pressure waves that are produced by the blood pumping action of the heart. The term cardiac cycle may be used to generally describe the series of events that occur during each heartbeat. The cardiac cycle may be divided into two general phases (diastole and systole). During diastole, the ventricles are relaxing and the ventricular chambers are filling with blood. During systole, the ventricles are contracting and ejecting blood out of the ventricular chambers, into the vasculature of the body. In some exemplary embodiments of a patient therapy system, a pulse generator of a BAT device delivers a burst of baroreflex therapy when the BAT enable signal assumes a high logic value. Also in these exemplary embodiments, the pulse generator is disabled when the BAT enable signal assumes a low logic value. In this way, the BAT enable signal can be used to synchronize the delivery of BAT bursts during a preferred phase of the cardiac cycle. With reference to FIG. 10, it will be appreciated that the BAT enable signal assumes a high logical value during each of the high pressure waves in arterial pressure waveform 800. The exemplary method illustrated in FIG. 10 may mimic the body's natural behavior. Normally, the baroreflex system is activated by pressure pulses passing through vasculature shortly after the heart contracts. Accordingly, the exemplary method illustrated in FIG. 10 activates the baroreflex system at a time when the body expects that baroreflex system to be activated. Timing the delivery of baroreflex activation therapy pulses in this way may increase the efficacy of the baroreflex activation therapy.

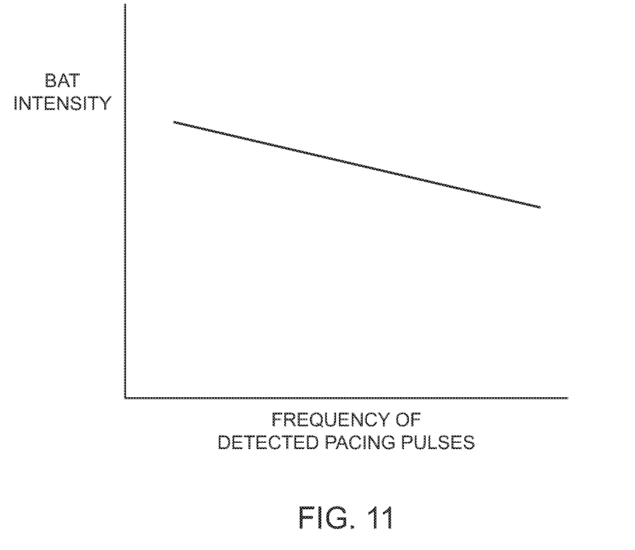

FIG. 11 is a graph of baroreflex activation therapy intensity as a function of heart rate. In some methods in accordance with the present invention, heart rate may be determined by detecting pacing pulses produced by a pacemaker and calculating the frequency of those pulses. In other methods, heart rate may be determined by detecting contractions of the heart and calculating the frequency of those contractions. A BAT device in accordance with some exemplary embodiments may detect heart contractions by measuring electrical potential differences between different locations within the body of the patient. When this is the case, the BAT device may repeatedly measure and record electrical potential differences to obtain a digitized electrocardiogram waveform. By analyzing these measured values and/or the electrocardiogram waveform, the BAT device can identify an R-wave portion of each cardiac cycle. The BAT device may determine heart rate, for example, by measuring the time interval between the peak of one R-wave and the peak of another R-wave.

After determining heart rate, the BAT device may use that information to adjust the intensity of the baroreflex activation therapy. In the exemplary embodiment of FIG. 11, BAT intensity is generally reduced as heart rate increases. BAT intensity can be changed, for example, by changing various characteristics of the BAT signal. Examples of signal characteristics that may be changed include duty cycle, pulse amplitude, pulse width, pulse frequency, pulse separation, pulse waveform, pulse polarity, and pulse phase. For further details of exemplary baroreflex activation devices, reference may be made to: U.S. Pat. Nos. 6,522,926 and 6,616,624; and U.S. patent application Ser. Nos. 09/964,079, 09/963,777, 09/963,991, 10/284,063, 10/453,678, 10/402,911, 10/402,393, 10/818,738, and 60/584,730, which were previously incorporated by reference.

Although the above description provides a complete and accurate representation of the invention, exemplary embodiments of the present invention may be manifested in a variety of forms other than the specific embodiments described and contemplated herein. Accordingly, departures in form and detail may be made without departing from the scope and spirit of exemplary embodiments of the present invention as described in the appended claims.

What is claimed is:

1. A method for treating a patient, comprising:
stimulating a baroreflex system of the patient with a baroreflex activation device according to a baroreflex activation therapy (“BAT”);
detecting a pacing pulse generated from a cardiac management device (“CRM”) device by the baroreflex activation device; and
adjusting the baroreflex activation therapy in response to the detected pacing pulse.

2. The method of claim 1, wherein the baroreflex activation device is connectable to an output terminal of the pacing pulse generator CRM device.

3. The method of claim 1, wherein the baroreflex activation device adjusts the timing of at least one baroreflex stimulation pulse based on detection of the detected pacing pulse.

4. The method of claim 3, wherein the baroreflex activation device comprises a pulse generator for providing at least one baroreflex stimulation pulse.

5. The method of claim 3, wherein adjusting the BAT comprises changing one or more characteristics of pulses generated by the baroreflex activation device.

6. The method of claim 5, wherein the characteristics of the pulse generated by the baroreflex activation device includes any one or more of pulse amplitude, frequency, burst energy, and wave characteristics.

7. The method of claim 1, wherein the BAT is delivered at a pre-selected time after detecting pacing pulses generated by the CRM device.

8. The method of claim 1, wherein the stimulation comprises stimulating a nerve or receptor.

9. The method of claim 8, wherein the nerve or receptor is a baroreceptor or a nerve leading from a baroreceptor.

10. The method of claim 1, wherein the intensity of the BAT is adjusted in response to the frequency of the detected pacing pulse.

11. The method of claim 1, wherein the BAT is performed in multiple steps.

12. The method of claim 1, wherein there is a pre-determined time delay between applying the multiple steps of the BAT.

13. The method of claim 1, wherein the BAT is delivered during systole or diastole cardiac phases.

14. The method of claim 7, wherein the time delay decreases as the frequency of detected pacing pulses increases.

15. The method of claim 1, wherein the baroreflex activation device and the CRM device are implantable within the patient.

16. The method of claim 15, wherein the baroreflex activation device and the CRM device are combined in a single housing.

17. The method of claim 15, wherein the baroreflex activation device and the CRM device are each housed in a separate housing.

18. The method of claim 1, wherein the pacing pulse generated by the CRM device is responsive to at least one or more of heart rate, cardiac waveform, timing of arterial and/or ventricular contractions, venous or arterial pressure, venous or arterial volume, cardiac output, pressure and/or volume in one or more heart chambers, cardiac efficiency, cardiac impedance, and edema.

19. The method of claim 1, wherein the method further comprises treatment of hypertension as well as resynchronization of the patient's heart.

20. The method of claim 1, wherein the method further comprises treatment of hypertension cardiac arrhythmia.

21. A method for treating a patient, comprising:
stimulating a baroreflex system of the patient with a baroreflex activation device according to a baroreflex activation therapy (“BAT”) during systole and diastole cardiac phases;
determining the patient's response to the baroreflex stimulation during the systole and diastole cardiac phases.

22. The method of claim 21, further comprising determining during which of the cardiac phases the BAT provides better therapy to the patient.

23. The method of claim 21, further comprising selecting a pre-determined time delay between the BAT such that the therapy is delivered during the preferred cardiac phase of systole or diastole.

24. The method of claim 21, further comprising detecting a pacing pulse generated from a CRM device by the baroreflex activation device.

25. The method of claim 24, wherein the baroreflex activation device is connectable to an output terminal of the CRM device.

26. The method of claim 24, further comprising adjusting the baroreflex system therapy in response to the detected pacing pulse.

27. The method of claim 23, wherein BAT is delivered after the pre-determined time delay.

28. The method according to claim 26, wherein the adjusting step as necessary is performed in a continuous loop to maintain effective BAT.

29. A method for treating a patient, comprising:
stimulating a baroreflex system of the patient according to a baroreflex activation therapy during the systole and the diastole of the cardiac phases; and
measuring the patient's response to the stimulation during the systole and diastole cardiac phases; and
determining which of the systole and diastole cardiac phases is more responsive to the baroreflex activation therapy (“BAT”) and is the preferred cardiac phase for baroreflex stimulation.

30. The method of claim 29, wherein BAT is delivered after a pre-determined time delay such that BAT is delivered during the preferred cardiac phase of systole or diastole.

31. The method of claim 29, further comprising:
stimulating the baroreflex system of the patient according to a therapy during the preferred cardiac phase; and
detecting by the baroreflex activation device a pacing pulse generated from a CRM device.

32. The method of claim 29, wherein the baroreflex activation device is connectable to an output terminal of the CRM device, the method further comprising continuously detecting the pacing pulse.

33. The method of claim 31, further comprising adjusting the baroreflex system therapy in response to the detected pacing pulse.

34. A method for treating a patient, comprising:
stimulating a baroreflex system of the patient according to a BAT deliverable by a baroreflex activation device being connected to an output terminal of a pacing pulse generator of a CRM device;
establishing a pre-determined time delay period prior to delivery of the BAT;
monitoring the pacing pulse during a pre-determined control time period: and
monitoring by the baroreflex activation device the output terminal of the CRM device for pacing pulse activity.

35. The method of claim 34, wherein the method further comprises delivering the baroreflex therapy upon expiration of the time delay and detection of pacing pulse.

36. The method of claim 34, wherein the baroreflex therapy is delivered even if no pacing pulse is detected after expiration of the controlled time period.

37. The method of claim 34, wherein the baroreflex activation device is connectable to an output terminal of the pacing CRM device, the method further comprising continuously monitoring of CRM device for generation of pacing pulse, until the baroreflex activation device detects a pacing pulse.

38. The method of claim 37, wherein the time delay is based on determining which of the systole or diastole cardiac phases is more responsive to the BAT.

39. A baroreflex activation system for treating a patient, comprising:
a baroreflex activation device connectable electrically connectable to an output terminal of a CRM device; and the baroreflex activation device including detecting circuitry for detecting at least one electrical pulse generated by the CRM device.

40. The system of claim 39, wherein the baroreflex activation device is programmable to apply a BAT including a plurality of therapy regimens.

41. The system of claim 39, wherein the CRM device comprises any one or more of a cardiac pacemaker, an implantable defibrillator, a cardioverter, a cardiac resynchronization device, or a combination device thereof.

42. The system of claim 41, wherein the pacing pulse is generated by a cardiac pacemaker.

43. The system of claim 41, wherein the pacing pulse is generated by an integrated pacemaker/defibrillator.

44. The system of claim 39, wherein the baroreflex activation device and the CRM device are housed in separate housings.

45. The system of claim 39, wherein the baroreflex activation device and the CRM device are housed in a single housing.

46. A method for treating a patient, comprising:
detecting a contraction in a heart of the patient; and
activating a baroreflex system of the patient after expiration of a pre-selected period of time.

47. The method of claim 46, wherein detecting a contraction in the heart of the patient comprises detecting electrical activity in the heart.

48. The method of claim 47, wherein detecting a contraction in the heart of the patient comprises detecting an R-wave in an electrocardiogram signal from the heart of the patient.

49. The method of claim 46, wherein detecting a contraction in the heart comprises detecting an electric pulse generated by a CRM device.

50. A method for treating a patient, comprising:
detecting an electric pulse generated by a CRM device; and
activating a baroreflex system of the patient after expiration of a pre-selected period of time.

51. The method of claim 50, wherein the CRM device comprises a pacemaker.

52. The method of claim 50, wherein the CRM device comprises a cardiac resynchronization therapy (CRT) device.

53. The method of claim 50, wherein the CRM device comprises a cardioverter.

54. The method of claim 50, wherein detecting the electric pulse generated by the CRM device comprises measuring a voltage difference between a BAT electrode and a housing of a BAT device.

55. The method of claim 50, wherein detecting the electric pulse generated by the CRM device comprises measuring a voltage difference between a lead of the CRM device and a housing of a BAT device.

56. The method of claim 50, wherein detecting the electric pulse generated by the CRM device comprises measuring a voltage difference between a lead of the CRM device and an electrode of a BAT device.

57. A method for treating a patient, comprising:
identifying a preferred phase of the patient's cardiac cycle for delivery of BAT to the patient's baroreflex system; and
activating the baroreflex system of the patient during the preferred phase of the patient's cardiac cycle.

58. The method of claim 57, wherein identifying a preferred phase of the patient's cardiac cycle comprises:
stimulating the baroreflex system of the patient during a systolic phase of the patient's cardiac cycle;
quantifying a first response based on the patient's response to the baroreflex stimulation during the systolic phase;
stimulating the baroreflex system of the patient during a diastolic phase of the patient's cardiac cycle;
quantifying a second response based on the patient's response to the baroreflex stimulation during the diastolic phase; and
comparing the second response to the first response.

59. The method of claim 58, further comprising identifying the preferred phase for the delivery of the BAT.

60. The method of claim 58, further comprising selecting the diastolic phase as the preferred phase if the second response is greater than the first response.

61. The method of claim 58, further comprising selecting the systolic phase as the preferred phase if the first response is greater than the second response.

62. A method for treating a patient, comprising:
detecting ventricular fibrillation in a heart of the patient;
defibrillating the heart of the patient; and
activating a baroreflex system of the patient to reduce the likelihood of the occurrence of ventricular fibrillation.

63. A method for treating a patient, comprising:
detecting an electric pulse generated by a CRM device; and
activating a baroreflex system of the patient in response to the detected electric pulse.

64. The method of claim 63, wherein the CRM device comprises a defibrillator.

65. The method of claim 63, further comprising discontinuing the activation of the baroreflex system of the patient after expiration of a pre-selected period of time.

66. A method for treating a patient, comprising:
delivering baroreflex activation therapy to a body of the patient;
detecting an electrical pulse generated by a CRM device; and
increasing an intensity of the baroreflex activation therapy upon detection of the electrical pulse.

67. The method of claim 66, wherein the CRM device comprises an implantable defibrillator.

68. The method of claim 66, further comprising returning the baroreflex activation therapy to an original intensity after expiration of a pre-selected period of time.

69. A method for treating a patient, comprising:
delivering baroreflex activation therapy to a body of the patient at a first intensity;
detecting an electrical pulse generated by a CRM device; and
delivering baroreflex activation therapy to the body of the patient at a second intensity after the electrical pulse has been detected.

70. The method of claim 69, wherein the second intensity is greater than the first intensity.

71. The method of claim 69, wherein the CRM device comprises an implantable defibrillator.

72. The method of claim 69, further comprising delivering baroreflex activation therapy to the body of the patient at the first intensity after expiration of a pre-selected period of time.
